(19) United States
(12) Patent Application Publication
(10) Pub. No.: US 2013/0023496 A1
(43) Pub. Date: Jan. 24, 2013

(54) Combination Therapy Comprising A CCR5 Antagonist, A HIV-1 Protease Inhibtior and a Pharmacokinetic Enhancer

(71) Applicants: Randy Tressler, Forest Hill, MD (US); Hernan Valdez, New York, NY (US)

(72) Inventors: Randy Tressler, Forest Hill, MD (US); Hernan Valdez, New York, NY (US)

(21) Appl. No.: 13/638,999

(22) PCT Filed: Mar. 30, 2011
(86) PCT No.: PCT/IB2011/051370

§ 371 Date: Oct. 2, 2012

Related U.S. Application Data
(60) Provisional application No. 61/320,428, filed on Apr. 2, 2010.

(51) Int. Cl. A61K 31/675; A61P 31/14; A61K 31/427; A61P 31/18; A61K 31/439; A61K 31/4402

(52) U.S. Cl. 514/81; 514/304; 514/357; 514/365

(57) ABSTRACT

The present invention discloses a novel combination therapy for HIV-1 treatment relying on a combination of at least one CCR5 antagonist, at least one HIV-1 protease inhibitor and at least one pharmacokinetic enhancer of said at least one CCR5 antagonist and/or at least one HIV-1 protease inhibitor. The combination is intended for use in oral treatment of a disorder selected from the group consisting of HIV-1 infection, retroviral infections genetically related to HIV and AIDS, in a treatment-naïve patient infected with CCR5 tropic HIV-1 virus.

### FIELD OF THE INVENTION

The present invention relates to a novel combination therapy for CCR5 tropic HIV-1 infected treatment-naïve patients.

### BACKGROUND OF THE INVENTION

Conventional regimens for HIV-1 infected treatment-naïve patients are based on multi-drug cocktails which usually encompass two NRTIs and a third drug, typically a non nucleoside reverse transcriptase inhibitor (NNRTI) or a protease inhibitor (PI). A typical example of such regimens is Atripla®, a fixed-dose combination containing efavirenz—a NNRTI—600 mg, emtricitabine—a NRTI—200 mg, and tenofovir disoproxil fumarate—an HIV-1 nucleotide analogue reverse transcriptase inhibitor (nRTI)—300 mg. Another example is the combination tested in the international, multi-center, open-label, non-inferiority, 96-week CASTLE study, wherein 440 treatment-naïve patients were randomized to receive Reyataz® (atazanavir sulphate)—an HIV-1 protease inhibitor (PI)—300 mg and ritonavir—another HIV-1 PI—100 mg once daily and 443 patients were randomized to receive co-formulated lopinavir—another HIV-1 PI—400 mg and ritonavir 100 mg twice daily. Both atazanavir/r and lopinavir/r were combined with a once-daily dose of Truvada®, i.e. a fixed-dose combination of emtricitabine 200 mg and tenofovir disoproxil fumarate 300 mg. The atazanavir arm met the non-inferiority primary endpoint of achieving undetectable viral load (defined as HIV-1 RNA less than 50 copies/mL) at 48 weeks.

An HIV-1 NRTI-sparing regimen for treatment-naïve patients would be desirable due to the concerns existing in the field about nucleoside analogues toxicities. Additionally, broadening the options for treatment-naïve patients by providing regimens containing drugs belonging to additional or different therapeutical classes than the current regimens would be beneficial in that the risk of viral resistance would be lowered and the options to manage patients with nucleoside-resistant virus would be broadened.

However, as of today, the relative contributions of each drug comprised in the known cocktails remains uncertain and it is impossible to predict whether e.g. removing the NRTIs from the existing combinations would disrupt a synergistic activity or a favourable complementary resistance profile. These aspects cannot be elucidated by studies in healthy volunteers since it is not simply a question of safety but uncertainties also exist as to the reaction of the HIV-1 virus to the treatment.

The central role of chemokine receptor CCR5 to the pathophysiology of inflammatory and infectious diseases is known. CCR5 co-receptor antagonists were found to inhibit fusion of HIV-1 with the host cell by blocking the interaction between the gp-120 viral glycoprotein and the CCR5 chemokine receptor on the host cell. In this manner, CCR5 antagonists are able to prevent infection of the cell and represent a valid mechanism for the treatment of HIV. Numerous disclosures in the art provide different chemical classes of CCR5 receptor modulators. A representative disclosure is Mills et al. WO 98/25617 relating to substituted aryl piperazines as modulators of chemokine receptor activity. Further disclosures are WO 98/025605; WO 98/025604; WO 98/002151; WO 98/004554; and WO 97/024325, WO 00/38680 and WO N-{(1S)-3-[3-(3-isopropyl-5-methyl-4H-1,2,4-triazol-4-yl)-exo-8-azabicyclo[3.2.1]oct-8-yl]-1-phenylpropyl}-4, 4-difluorocyclohexanecarboxamide, also known as maraviroc, is disclosed in EP-A-1284974; see in particular examples 4, 6 and 7. Maraviroc is the first, orally available, approved medication belonging to the class of CCR5 antagonists. Like all CCR5 antagonists, maraviroc blocks the entry of HIV-1 virus exclusively through the CCR5 co-receptor. Consequently, maraviroc can be defined as a selective CCR5 antagonist and is useful for treating patients infected with CCR5 tropic version of the HIV-1 virus.

CCR5 antagonists like maraviroc are generally associated with few side effects, a significant increase in CD4+ cell counts, and seem to play a role in reducing inflammation, which may offer benefits beyond viral load suppression. Additionally, it has been noted that treatment naïve patients tend to be more frequently infected with CCR5-tropic virus, which is sensitive to CCR5 antagonists. Accordingly, using MVC at an early stage of the treatment could also be beneficial in that other options would be preserved for later stages. Finally, CCR5 antagonists are the only anti HIV class of drugs to which HIV virus can lose sensitivity without having been exposed to the drug. Thus, if one used other drugs first, clinicians might loose the therapeutical option of using CCR5 antagonists. For these and other reasons, it would be desirable to include CCR5 antagonists in regimens for treatment-naïve patients.

Efficacy of maraviroc in HIV-1 treatment naïve patients is investigated in the MERIT study. This trial is a randomized, double-blind clinical study designed to compare the safety and efficacy of maraviroc 300 mg, twice per day (BID) versus efavirenz (a non-nucleoside reverse transcriptase inhibitor (NNRTI)) 600 mg, once per day (QD), both orally administered with zidovudine and lamivudine (two NRTIs), in treatment-naïve patients infected with CCR5 tropic HIV-1 virus. The MERIT study did not investigate other cocktails and notably NRTI-sparing cocktails as well as the related dosage regimens. Additionally, preliminary results highlighted that the maraviroc 300 mg QD arm did not meet the regulatory prespecified criteria for non-inferiority versus the efavirenz arm. As a consequence, the MVC QD arm was terminated.

Other maraviroc-containing regimens were further investigated in numerous DDI (drug-drug interaction) studies carried out in healthy volunteers. For example, open-label, randomized, placebo-controlled studies conducted in healthy subjects assessed the effect of separate and distinct combinations of CYP3A4 inhibitors (such as HIV-1 protease inhibitors) on the steady-state pharmacokinetics of maraviroc (“Effects of CYP3A4 inhibitors on the pharmacokinetics of maraviroc in healthy volunteers”, S. Abel et al., Br J Clin Pharmacol. 2008 April; 65 Suppl. 1:27-37). As highlighted above, DDI studies are limited to verifying safety in healthy volunteers. Hence, they are not a suitable basis to elucidate whether the tested cocktails are effective to treat an HIV-1 infected treatment-naïve patient and define the corresponding therapeutically effective dosage regimens.

Finally, the wish to test efficacy of novel, once per day, CCR5-containing regimens in treatment-naïve patients is known in the field of HIV treatment. However, as of today, no data is available about efficacy, tolerability, durability, convenience, required dosing, pharmacokinetic profile, medication adherence, resistance and general safety of such novel therapies in HIV-1 infected patients. Hence, apart from the multidrug cocktail tested in the MERIT study, the provision of an alternative CCR5-containing safe, effective and affordable antiretroviral drug regimen for treatment naïve HIV-1 infected patients remains today a mere desire.

An object of the present invention is thus to provide a novel CCR5-containing regimen for treatment-naïve patients infected with CCR5 tropic HIV-1 virus that inter alia ensures safety, efficacy and minimal side effects as well as a simplified dose regimen and an enhanced adherence.

### SUMMARY OF THE INVENTION

The above and other objects are achieved by a combination comprising a therapeutically effective amount of at least one CCR5 antagonist, at least one HIV-1 protease inhibitor and at least one pharmacokinetic enhancer of said at least one CCR5 antagonist and/or at least one HIV-1 protease inhibitor for use in oral treatment of a disorder selected from the group consisting of HIV-1 infection, retroviral infections genetically related to HIV, and AIDS, in a treatment-naïve patient infected with CCR5 tropic HIV-1 virus.

The above and other objects are also achieved by the use of a combination comprising a therapeutically effective amount of at least one CCR5 antagonist, at least one HIV-1 protease inhibitor and at least one pharmacokinetic enhancer of said at least one CCR5 antagonist and/or at least one HIV-1 protease inhibitor for the manufacture of a medicament for oral treatment of a disorder selected from the group consisting of HIV-1 infection, retroviral infections genetically related to HIV, and AIDS, in a treatment-naïve patient infected with CCR5 tropic HIV-1 virus.

The above and other objects are also achieved by a pharmaceutical composition for oral administration to a treatment-naïve patient infected with CCR5 tropic HIV-1 virus, said composition comprising a therapeutically effective amount of at least one CCR5 antagonist, at least one HIV-1 protease inhibitor, at least one pharmacokinetic enhancer of said at least one CCR5 antagonist and/or at least one HIV-1 protease inhibitor and one or more pharmaceutically acceptable excipients, carriers and/or diluents.

The above and other objects are achieved by a unit dosage form comprising a therapeutically effective amount of at least one CCR5 antagonist, at least one HIV-1 protease inhibitor and at least one pharmacokinetic enhancer of said at least one CCR5 antagonist and/or at least one HIV-1 protease inhibitor for use in oral treatment of a disorder selected from the group consisting of HIV-1 infection, retroviral infections genetically related to HIV-1 and AIDS, in a treatment-naïve patient infected with CCR5 tropic HIV-1 virus.

The above and other objects are also achieved by a kit comprising a therapeutically effective amount of at least one CCR5 antagonist, at least one HIV-1 protease inhibitor and at least one pharmacokinetic enhancer of said at least one CCR5 antagonist and/or at least one HIV-1 protease inhibitor as a combined preparation for simultaneous, separate or sequential oral administration to a treatment-naïve patient infected with CCR5 tropic HIV-1 virus.

The above and other objects are achieved by a method of treating a disorder selected from the group consisting of HIV-1 infection, retroviral infections genetically related to HIV, and AIDS, said method comprising simultaneous, separate or sequential oral administration of a therapeutically effective amount of at least one CCR5 antagonist, at least one HIV-1 protease inhibitor and at least one pharmacokinetic enhancer of said at least one CCR5 antagonist and/or at least one HIV-1 protease inhibitor to a treatment-naïve patient infected with CCR5 tropic HIV-1 virus.

### FIGURES

FIG. 1 is a line plot of median HIV-1 RNA (log10 copies/mL) by visit. The parameter is reported for a combination of the invention (maraviroc 150 mg QD and atazanavir 300 mg/ ritonavir 100 mg QD) and a reference combination (atazanavir sulphate 300 mg and ritonavir 100 mg both QD+emtricitabine 200 mg and tenofovir disoproxil fumarate 300 mg, both QD). N1 and N2 are number of subjects for maraviroc+atazanavir/ritonavir and atazanavir/ritonavir+emtricitabine/tenofovir, respectively at each point in time. Baseline is calculated as mean of screening, randomization and day 1 measurement before starting the treatment. One subject from sub-analysis was off by one day on day 7 and day 14, this subject is included in the analysis for day 7, day 14;

FIG. 2 is a line plot of median HIV-1 RNA change from baseline (log10 copies/mL) by visit. The same comments made above for FIG. 1 apply;

FIG. 3 is a line plot of median CD4 cell count by visit. Like for FIG. 1., two curves are reported: one for a combination of the invention (maraviroc 150 mg QD and atazanavir 300 mg/ ritonavir 100 mg QD) and one for a reference combination (atazanavir sulphate 300 mg and ritonavir 100 mg both QD+emtricitabine 200 mg and tenofovir disoproxil fumarate 300 mg, both QD). N1 and N2 are number of subjects for maraviroc+atazanavir/ritonavir and atazanavir/ritonavir+emtricitabine/tenofovir, respectively at each time in point. Baseline is calculated as mean of screening, randomization and day 1 measurement before starting the treatment;

FIG. 4 is a line plot of median CD4 cell count change from baseline by visit. The same comments made above for FIG. 3 apply;

FIG. 5 is a line plot of median CD8 cell count by visit. Like for FIG. 1., two curves are reported: one for a combination of the invention (maraviroc 150 mg QD and atazanavir 300 mg/ritonavir 100 mg QD) and one for a reference combination (atazanavir sulphate 300 mg and ritonavir 100 mg both QD+emtricitabine 200 mg and tenofovir disoproxil fumarate 300 mg, both QD). N1 and N2 are number of subjects for maraviroc+atazanavir/ritonavir and atazanavir/ritonavir+emtricitabine/tenofovir, respectively at each time in point. Baseline is calculated as mean of screening, randomization and day 1 measurement before starting the treatment;

FIG. 6 is a line plot of median CD8 cell count change from baseline by visit. The same comments made above for FIG. 5 apply.

### DETAILED DESCRIPTION OF THE INVENTION

Below, features and embodiments of the present invention are discussed. When reference is made to one aspect of the present invention, only—e.g. the combination—it must be understood that unless otherwise indicated, the same features and embodiments are, to the possible technical extent, also applicable to all other aspects of the invention—i.e. use of such combination, a pharmaceutical composition comprising such combination, a kit comprising such combination, a unit dosage form comprising such combination and a method of treatment by administering such combination.

In one embodiment, “pharmacokinetic enhancer of said at least one CCR5 antagonist and/or at least one HIV-1 protease inhibitor” (hereinafter also “pharmacokinetic enhancer”) preferably means a drug that, by inhibiting the metabolic pathway of said at least one CCR5 antagonist and/or said at least one HIV-1 protease inhibitor, preferably both, decreases their metabolic clearance (this is also referred to in the present application as “boost” or “boosting activity”).

In one embodiment, the at least one pharmacokinetic enhancer is at least one CYP3A4 inhibitor. In vitro assays to evaluate in a predictive manner in vivo CYP3A4 inhibition of a named drug are known in the field of pharmaceuticals. Reference can be made for example to comments and assays discussed in “Guidance for Industry—Drug interaction Studies—Study Design, Data Analysis, and Implications for Dosing and labelling—Draft Guidance—September 2006”, in particular Appendix C1, C2 and C3. An electronic copy of this document is available from US FDA website. In one embodiment, the CYP3A4 inhibition activity may be the only pharmacological effect or one of multiple and pharmacologically distinct effects of said at least one pharmacokinetic enhancer. In one embodiment, said at least one CYP3A4 inhibitor is a drug that causes a 5-fold increase in the plasma AUC values or more than 80% decrease in clearance of CYP3A substrates in clinical evaluations. Typical CYP3A substrates may be selected from the group comprising midazolam, buspirone, felodipine, lovastatin, eletriptan, sildenafil, simvastatin and triazolam. Definition of CYP3A4 inhibitor and CYP3A substrates are as per USA FDA “Drug Development and Drug Interactions: Table of Substrates, Inhibitors and Inducers” (i) Table 5—Classification of CYP3A inhibitors—strong CYP3A inhibitor and (ii) Table 4—Examples of in vivo substrate, inhibitor, and inducer for specific CYP enzymes for study (oral administration), respectively.

In one embodiment, said at least one pharmacokinetic enhancer comprises, preferably consists of cobicistat or a pharmaceutically acceptable salt or solvate thereof.

In one embodiment, said at least one pharmacokinetic enhancer comprises, preferably consists of a second HIV-1 protease inhibitor which is different over the at least one HIV-1 protease inhibitor. Accordingly, in this embodiment, the combination of the invention comprises at least one CCR5 antagonist and at least two HIV-1 protease inhibitors. Unless otherwise indicated, references below to “at least two HIV-1 protease inhibitors” refer to this embodiment.

In one embodiment, “treatment-naïve patient” means a patient infected with a CCR5 tropic HIV-1 virus who has not received any previous HIV drug therapy. In this embodiment, the language treatment-naïve patient is used in juxtaposition to patients who have received some HIV drug therapy and that will eventually be referred to as treatment experienced patients.

In one embodiment, “treatment-naïve patient” means a patient infected with a CCR5 tropic HIV-1 virus who has not received any previous HIV-1 protease inhibitor and/or CCR5 antagonist drug containing therapy. In this embodiment then, a CCR5 tropic HIV-1 infected patient is naïve to HIV-1 protease inhibitors and/or CCR5 antagonists but may have already been administered with one or more other HIV-1 therapy drugs.

In one embodiment, a treatment-naïve patient is preferably an adult treatment-naïve patient.

Different strains of HIV-1 virus exist which are (mainly) distinguished according to the CD4+ and T-cells co-receptors used by the virus to gain entry into the host cell. Macrophage (M-tropic) strains of HIV-1 or non-syncitia-inducing strains (NSI) use the beta-chemokine receptor CCR5. T-tropic strains of HIV-1 or syncitia-inducing (SI) strains use the alpha-chemokine receptor, CXCR4, for entry. HIV-1 strains also exist that are able to indifferently use CCR5 and CXCR4 co-receptors (dual tropic strains). A HIV-1 viral population containing substantially CCR5-using virus is generally classified as CCR5 tropic HIV-1 virus. A HIV-1 viral population containing substantially CXCR4-using virus is generally classified as CXCR4 tropic HIV-1 virus. A HIV-1 viral population containing both CCR5-using and CXCR4-using virus is generally classified as mixed tropic HIV-1 virus. A HIV-1 viral population that can enter host CD4+ cells via either the CCR5 or CXCR4 co-receptors is generally classified as dual tropic virus HIV-1 virus.

In the context of the present invention, “CCR5 tropic HIV-1 virus” preferably refers to a viral population containing detectable amounts of CCR5 tropic HIV-1 virus, preferably at least 20%, more preferably at least 50%, more preferably at least 80%, even more preferably at least 95% of CCR5 tropic HIV-1 virus over the total amount of HIV-1 virus present.

Methods are known in the art to identify the HIV-1 viral population (i.e. HIV-1 tropism) in a patient. A commercial method is the Trofile® molecular assay developed by Monogram Biosciences for use in HIV treatment. The assays results show whether the patient is infected with virus that enters cells using the CCR5 co-receptor, the CXCR4 co-receptor, or both (dual/mixed).

Unless otherwise indicated, “treatment” preferably encompasses improving one or more of the following parameters in a treatment-naïve patient: (i) HIV-1 RNA copies and (ii) CD4+ cell count. Preferably both parameters (i) and (ii) are improved. Unless otherwise indicated, improving one or more of the parameters mentioned above means ameliorating the value of the named parameter over the baseline value of the same parameter calculated in the same patient. The baseline value is calculated as the mean of the values of the named parameter in a patient at patient screening visit, patient randomization and day 1 before starting the treatment.

In one embodiment, an improvement of one or more of the parameters mentioned above is obtained after at least two (2) weeks, more preferably after at least seven (8) weeks, even more preferably after at least twenty-four (24) weeks of treatment.

In one embodiment, improving HIV-1 RNA copies in a patient means obtaining a reduction of HIV-1 RNA copies over baseline. In one embodiment, the improvement can be calculated as viral load, i.e. absolute HIV-1 RNA copies/mL of patient's blood plasma. In this embodiment, improving HIV-1 RNA copies preferably means obtaining a viral load of less than 400 copies/mL, preferably less than 50 copies/mL. In another embodiment, the improvement can be calculated as log₁₀ copies of HIV-1 RNA/mL of patient's blood plasma over baseline. In this embodiment, the reduction is preferably of at least 1.5 log₁₀ copies, more preferably of at least 2.0 log₁₀ copies, even more preferably at least between 2.0 and 3.0 log₁₀ copies.

In one embodiment, improving CD4+ cell count in a patient means increasing the median CD4+ cell count over baseline. The improvement can be calculated as median CD4+ cells/μL of patient blood plasma over baseline. Preferably, the increase is of at least 50 CD4+ cells/μL, more preferably of at least 100 CD4+ cells/μL, even more preferably of at least 150 CD4+ cells/μL, even more preferably of at least 200 CD4+ cells/μL.

As a consequence of the treatment according to the present invention, a reduction is obtained in the risk for a treatment naïve patient of contracting an HIV-1 related opportunistic condition and/or an improvement is obtained in the patient's ability to fight existing HIV related opportunistic conditions. HIV related opportunistic conditions include opportunistic infections and malignancies. Examples of HIV related opportunistic conditions include pneumocystitis carinii, toxoplasmosis, isoporiasis, cryptosporidiosis, candidiasis, cryptococcosis, histoplasmosis, coccidioidomycosis, mycobacterium tuberculosis, non tuberculosis mycobacterium infections, salmonella, cytomegalovirus, herpes simplex virus, recurrent or persistent upper respiratory infection, sinuisitis, otitis media, bacterial meningitis, pneumonia, sepsis, oropharyngis candidaiasis, diarrhea, hepatitis, herpes zoster, leiomyosarcoma, lymphoid interstiticial pneumonia, nocardiosis, disseminated varicella, and toxoplasmosis of the brain, progressive multifocal leukoencephalopathy, Kaposi's sarcoma, lymphoma, cervical cancer, HIV dementia and HIV wasting syndrome.

Unless otherwise indicated, retroviral infections genetically related to HIV-1 means infections caused by viruses related to HIV and that use the same or similar enzymes used by HIV to complete their life cycle. Preferably, retroviral infections genetically related to HIV-1 are selected from the group consisting of all subclasses of HIV-1, HIV-2 and other related retroviruses.

In one embodiment, the combination of the invention does not contain HIV-1 nucleotide reverse transcriptase inhibitors (NRTIs). In one embodiment, the combination of the invention does not contain HIV-1 nucleoside reverse transcriptase inhibitors (nRTIs). In one embodiment, the combination of the invention does not contain HIV-1 integrase inhibitors (e.g. raltegravir). In one embodiment, the combination of the invention does not contain HIV-1 NRTIs, nRTIs and integrase inhibitors.

In one embodiment, the combination of the invention does not contain HIV-1 therapy drugs other than at least one CCR5 antagonist, at least one HIV-1 protease inhibitor and at least one pharmacokinetic enhancer. In one embodiment, the combination of the invention does not contain HIV-1 therapy drugs other than at least one CCR5 antagonist and at least two HIV-1 protease inhibitors. Unless otherwise indicated, HIV-1 therapy drugs mean drugs active at least in vivo on a HIV-1 infection, retroviral infections genetically related to HIV and AIDS. Activity on HIV-1 infection, retroviral infections genetically related to HIV and AIDS (hereinafter also “anti HIV-1 activity”) preferably comprises direct activity (i.e. a drug displays per se anti HIV-1 activity) and indirect activity (i.e. a drug does not display per se anti HIV-1 activity but boosts direct anti HIV-1 activity of one or more other drugs contained in the composition). In one embodiment, anti HIV-1 activity is direct activity. In a different embodiment, anti HIV-1 activity is indirect activity. In one embodiment, anti HIV-1 activity may be the only pharmacological effect of HIV-1 therapy drugs or one of multiple and pharmacologically distinct effects of HIV-1 therapy drugs. HIV-1 therapy drugs displaying direct anti HIV-1 activity are often classified in therapeutical classes according to their structure or mechanism of action on HIV virus. For example, HIV-1 therapy drugs displaying direct anti HIV-1 activity include CCR5 antagonists, HIV-1 protease inhibitors, HIV-1 NNRTIs, HIV-1 NRTIs, HIV-1 integrase inhibitors, HIV-1 fusion inhibitors, HIV-1 maturation inhibitors and virostatics.

In one embodiment, the combination of the invention does not contain pharmaceutically active substances (as opposed to excipients) other than at least one CCR5 antagonist, at least one HIV-1 protease inhibitor and at least one pharmacokinetic enhancer.

In one embodiment, the combination of the invention consists of at least one CCR5 antagonist, at least one HIV-1 protease inhibitor and at least one pharmacokinetic enhancer of said at least one CCR5 antagonist and/or at least one HIV-1 protease inhibitor and, optionally, pharmaceutically active excipients, carriers and/or diluents.

Unless otherwise indicated, the wording CCR5 antagonists, HIV-1 protease inhibitors and pharmacokinetic enhancers comprises any free acid or base, or pharmaceutically acceptable salt, solvate or prodrug of any CCR5 antagonist, HIV-1 protease inhibitor and pharmacokinetic enhancers as defined herein.

Pharmaceutically acceptable salts of CCR5 antagonists, HIV-1 protease inhibitors and pharmacokinetic enhancers include the acid addition and base salts of thereof.

Suitable acid addition salts are formed from acids which form non-toxic salts. Examples include the acetate, aspartat, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, rotate, oxalate, palmitate, pamoate, phosphate/hydrogen phosphate/dihydrogen phosphate, saccharate, stearate, succinate, tartrate, tosylat and trifluoroacetate salts. An example is CCR5 antagonist salt is N-{(1S)-3-[3-endo-(5-Isobutyryl-2-methyl-4,5,6,7-tetrahydro-1-H-imidazo[4, 5-c]pyridin-1-yl)-8-azabicyclo-[3.2.1]oct-8-yl]-1-(3-fluorophenyl)propyl}acetamide fumarate, disclosed in PCT/IB2004/003153.

Suitable base salts are formed from bases which form non-toxic salts. Examples include the aluminium, arginin, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

A pharmaceutical acceptable salt of CCR5 antagonists, HIV-1 protease inhibitors and pharmacokinetic enhancers may be readily prepared by mixing together solutions of a named CCR5 antagonist, HIV-1 protease inhibitor and pharmacokinetic enhancer and the desired acid or base, as appropriate. The salt may precipitate from solution and be collected by filtration or may be recovered by evaporation of the solvent. The degree of ionisation in the salt may vary from completely ionised to almost non-ionised.

CCR5 antagonists, HIV-1 protease inhibitors and pharmacokinetic enhancers may exist in both unsolvated and solvate forms. The term ‘solvate’ is used herein to describe a molecular complex comprising CCR5 antagonists, HIV-1 protease inhibitors and pharmacokinetic enhancers and one or more pharmaceutically acceptable solvent molecules, for example, ethanol. The term ‘hydrate’ is employed when said solvent is water.

Unless otherwise indicated, the wording CCR5 antagonists, HIV-1 protease inhibitors and pharmacokinetic enhancers comprises any polymorphs and prodrugs thereof. Also included are crystalline or amorphous products. Such products may be obtained, for example, as solid plugs, powders, or films by methods such as precipitation, crystallization, freeze drying, spray drying, or evaporative drying. Microwave or radio frequency drying may be used for this purpose.

Also within the scope of the invention are so-called ‘prodrugs’ of CCR5 antagonists, HIV-1 protease inhibitors and pharmacokinetic enhancers. Certain derivatives of CCR5 antagonists, HIV-1 protease inhibitors and pharmacokinetic enhancers which may have little or no pharmacological activity themselves can, when administered into or onto the body, be converted into compounds having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs in accordance with the invention can, for example, be produced by replacing appropriate functionalities present in CCR5 antagonists, HIV-1 protease inhibitors and pharmacokinetic enhancers with certain moieties known to those skilled in the art as ‘pro-moieties’ as described, for example, in “Design of Prodrugs” by H Bundgaard (Elsevier, 1985).

For example, when a named CCR5 antagonist, HIV-1 protease inhibitor and pharmacokinetic enhancer contains an alcohol functionality (—OH), some examples of prodrugs in accordance with the invention may include an ester or ether thereof, for example, by replacement of the hydrogen by phosphorylation.

CCR5 antagonists, HIV-1 protease inhibitors and pharmacokinetic enhancers may contain one or more asymmetric carbon atoms and may therefore exist as two or more stereoisomers. Where for example a named CCR5 antagonist, HIV-1 protease inhibitor and pharmacokinetic enhancer contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the present invention are all stereoisomers, geometric isomers and tautomeric forms of CCR5 antagonists, HIV-1 protease inhibitors and pharmacokinetic enhancers, including compounds exhibiting more than one type of isomerism, and mixtures of one or more thereof. When a named CCR5 antagonist, HIV-1 protease inhibitor and pharmacokinetic enhancer contains a basic or acid moiety, also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the named CCR5 antagonist, HIV-1 protease inhibitor and pharmacokinetic enhancer contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer (s) by means well known to a skilled person.

Stereoisomeric conglomerates may be separated by conventional techniques known to those skilled in the art-see, for example “Stereochemistry of Organic Compounds” by E L Eliel (Wiley, N.Y., 1994).

Unless otherwise indicated, the at least one CCR5 antagonists of the invention are selective CCR5 antagonists (or inhibitors) in the sense that they are able to block the entry of HIV-1 virus into host cells like CD4+ cells, T-cells or macrophages by exclusively acting on the CCR5 chemokine co-receptor. No viral entry blocking effect is exerted by CCR5 antagonists on HIV-1 populations exclusively using CXCR4 co-receptor to infect host cells.

In one embodiment, the one or more CCR5 antagonists of the invention have an IC₅₀ for CCR5 co-receptor of less than 1 μM (as determined by the MIP-1βassay of Combadiere et al, J. Leukoc. Biol., 60, 147-152 (1996)). In one embodiment, the one or more CCR5 antagonists of the invention have an IC₅₀ for CXCR4 co-receptor of more than 10 μM (Dorr P. et al.; “Maraviroc (UK-427,857), a Potent, Orally Bioavailable, and Selective Small-Molecule Inhibitor of Chemokine Receptor CCR5 with Broad-Spectrum Anti-Human Immunodeficiency Virus Type 1 Activity”; Antimicrob Agents Chemother. 2005 November; 49(11): 4721-4732). These assays are both binding and functional ones, i.e. they can be used to identify CCR5 antagonism and discriminate among CCR5 antagonists based on their binding efficacy.

In a further embodiment, the at least one CCR5 antagonist of the invention is selected from the group consisting of maraviroc, NCB-9471, PRO-140, CCR5mAb004, TAK-779 (disclosed in WO 99/32468), ZM-688523, 4-chloro-6-fluoro sulphonamide,TAK-220 (disclosed in WO 01/25200), TAK-652 (disclosed in WO03014105 and having the chemical name 8-[4-(2-butoxyethoxy)phenyl]-1-isobutyl-N-[4-[[(1-propyl-1H-imadazol-5-yl)methyl]sulphinyl]phenyl]-1,2,3,4-tetrahydro-1-benzacocine-5-carboxamide), SC-351125, ancriviroc (formerly known as SCH-C), vicriviroc (which has the chemical name (4,6-dimethylprymidine-5-yl){4-[(3S)-4-{(1R)-2-methoxy-1-[4-(trifluoromethyl) phenyl]ethyl}-3-methylpiperazin-1-yl]-4-methylpiperidin-1-yl}methanone), PRO-140, aplaviroc (formerly known as GW-873140, Ono-4128, AK-602), AMD-887, INC-B9471, CMPD-167 (which has the chemical name N-methyl-N-((1R,3S,4S)-3-[4-(3-benzyl-1-ethyl-1H-pyrazol-5-yl) piperidin-1-ylmethyl]-4-[3-fluorophenyl]cyclopent-1-yl]-D-valine)), methyl1-endo-{8-[(3S)-3-(acetylamino)-3-(3-fluorophenyl) propyl]-8-azabicyclo[3.2.1]oct-3-yl}-2-methyl-4,5,6,7-tetrahydro-1H-imidazo[4, 5-c]pyridine-5-carboxylate, methyl 3-endo-{8-[(3S)-3-(acetamido)-3-(3-fluorophenyl)propyl]-8-azabicyclo [3.2.1]oct-3-yl}-2-methyl-4,5,6,7-tetrahydro-3H-imidazo[4,5-c]pyridine-5-carboxylate, ethyl 1-endo-{8-[(3S)-3-(acetylamino)-3-(3-fluorophenyl)propyl]-8-azabicyclo[3.2.1]oct-3-yl}-2-methyl-4, 5,6,7-tetrahydro-1H-imidazo[4,5-c]pyridine-5-carboxylate and N-{(1S)-3-[3-endo-(5-Isobutyryl-2-methyl-4,5,6, 7-tetrahydro-1H-imidazo[4,5-c]pyridin-1-yl)-8-azabicyclo[3.2.1]oct-8-yl]-1-(3-fluorophenyl)propyl}acetamide) and pharmaceutically acceptable salts, solvates or derivatives of the above. The last four compounds are disclosed in WO 03/084954 and WO 05/033107.

In a yet further embodiment, the one or more CCR5 antagonists are selected from the group consisting of maraviroc, vicriviroc, NCB-9471, PRO-140, CCR5mAb004, 8-[4-(2-butoxyethoxy)phenyl]-1-isobutyl-N-[4-[[(1-propyl-1 H-imadazol-5-yl)methyl]sulphinyl]phenyl]-1,2,3,4-tetrahydro-1-benzacocine-5-carboxamide, methyl 1-endo-{8-[(3S)-3-(acetylamino)-3-(3-fluorophenyl)propyl]-8-azabicyclo[3.2.1]oct-3-yl}-2-methyl-4, 5,6,7-tetrahydro-1H-imidazo[4,5-c]pyridine-5-carboxylate, methyl 3-endo-{8-[(3S)-3-(acetamido)-3-(3-fluorophenyl) propyl]-8-azabicyclo[3.2.1]oct-3-yl}-2-methyl-4,5,6,7-tetrahydro-3H-imidazo[4,5-c]pyridine-5-carboxylate, ethyl 1-endo-{8-[(3S)-3-(acetylamino)-3-(3-fluorophenyl)propyl]-8-azabicyclo[3.2.1]oct-3-yl}-2-methyl-4, 5,6,7-tetrahydro-1H-imidazo[4,5-c]pyridine-5-carboxylate, and N-{(1S)-3-[3-endo-(5-Isobutyryl-2-methyl-4, 5,6,7-tetrahydro-1H-imidazo[4,5-c]pyridin-1-yl)-8-azabicyclo[3.2.1]oct-8-yl]-1-(3-fluorophenyl) propyl}acetamide) and pharmaceutically acceptable salts, solvates or derivatives of the above.

In a yet further embodiment, the combination of the invention contains at least maraviroc as CCR5 antagonist.

In a further embodiment, the one or more CCR5 antagonists of the invention are only one CCR5 antagonist.

In a further embodiment, the one or more CCR5 antagonists of the invention are only one CCR5 antagonist which is maraviroc.

Protease inhibitors (PIs) are a known therapeutical class of drugs used to treat or prevent infection by e.g. HIV and Hepatitis C. HIV-1 PIs prevent viral replication by inhibiting the activity of HIV-1 protease, an enzyme used by the viruses to cleave nascent proteins for final assembly of new virons. Screening of protease inhibitors can be done according to methods and assays discussed in “J Rose and C Craik, Structure-assisted design of nonpeptide human immunodeficiency virus-1 protease inhibitors, Am J Respir Crit Care Med 150 (1994), pp. 5176-5182”.

In one embodiment, the combination of the invention comprises at least two HIV-1 protease inhibitors wherein one is ritonavir and the other is selected from the group consisting of lopinavir, atazanavir, fosamprenavir, darunavir and mixtures thereof, more preferably the group consisting of lopinavir, atazanavir, darunavir and mixtures thereof.

In one embodiment, at least one HIV-1 protease inhibitor is only one HIV-1 protease inhibitor.

In one embodiment, the combination of the invention comprises at least two HIV-1 protease inhibitors wherein one is atazanavir and the other is ritonavir (the combination of which is also referred to below as “atazanavir/r” or “ritonavir boosted atazanavir”).

In one embodiment, the combination comprises a therapeutically effective amount of maraviroc, atazanavir and ritonavir.

In one embodiment, the at least one CCR5 antagonist is administered according to a once per day (QD), or twice per day (BID) regime, preferably a once per day (QD) regime.

In one embodiment, the at least one HIV-1 protease inhibitor and at least one pharmacokinetic enhancer are administered, preferably concurrently, according to a once per day (QD) or twice per day (BID) regime, preferably a once per day (QD) regime.

In one embodiment, both the at least one CCR5 antagonist, the at least one HIV-1 protease inhibitor and at least one pharmacokinetic enhancer are administered, preferably concurrently, according to a once per day (QD) regime.

In one alternative embodiment, the at least one CCR5 antagonist is administered according to a once per day (QD) regimen whereas the at least one HIV-1 protease inhibitor and at least one pharmacokinetic enhancer are administered according to a twice per day (BID) regime.

In one embodiment, the at least one CCR5 antagonist is administered at an amount between about 150 and about 300 mg die (per day), more preferably at about 150 mg die.

In one embodiment, the at least one HIV-1 protease inhibitor and at least one pharmacokinetic enhancer are administered at an amount between about 400 and about 1600 mg die, preferably between about 400 and about 1000 mg die, more preferably at about 400 mg die.

In one embodiment, one of the at least one pharmacokinetic enhancer is ritonavir and it is administered at an amount between about 100 and about 200 mg die, preferably 100 mg die.

In one embodiment, one of at least one HIV-1 protease inhibitor is atazanavir and it is administered at an amount of about 300 mg die.

In one embodiment, the combination of the invention comprises at least one CCR5 antagonist at an amount between about 150 and about 300 mg and at least one HIV-1 protease inhibitor and at least one pharmacokinetic enhancer at an amount between about 400 and about 1600 mg.

In one embodiment, the combination of the invention comprises maraviroc at an amount between about 150 and about 300 mg, ritonavir at an amount between about 100 and about 200 mg and at least another HIV-1 protease inhibitor at an amount between about 300 mg and 800 mg.

In one embodiment, the combination of the invention comprises maraviroc at an amount between about 150 and about 300 mg, ritonavir at an amount between about 100 and about 200 mg and atazanavir at an amount of 300 mg.

In one embodiment, the combination of the invention comprises maraviroc 150 mg, atazanavir 300 mg and ritonavir 100 mg for use in oral treatment of a disorder selected from the group consisting of HIV-1 infection, retroviral infections genetically related to HIV, and AIDS, in a treatment-naïve patient infected with CCR5 tropic HIV-1 virus. In one embodiment, this combination is for use in a once per day (QD) regimen oral treatment.

In the combination of the present invention, the at least one CCR5 antagonist, the at least one HIV-1 protease inhibitor and the at least one pharmacokinetic enhancer may be administered, in terms of dosage forms, either separately or in conjunction with each other; and in terms of their time of administration, either simultaneously, separately or sequentially. For example, the administration of at least one CCR5 antagonist may be prior to, concurrent with, or subsequent to the administration of either one or both the at least one HIV-1 protease inhibitor and the at least one pharmacokinetic enhancer. In one embodiment, the at least one CCR5 antagonist, the at least one HIV-1 protease inhibitor and the at least one pharmacokinetic enhancer are administered simultaneously or sequentially, preferably simultaneously. In one embodiment, the at least one CCR5 antagonist, the at least one HIV-1 protease inhibitor and the at least one pharmacokinetic enhancer are administered separately. Preferably, the time between administration of the at least one CCR5 antagonist and the last administered of the at least one HIV-1 protease inhibitor and the at least one pharmacokinetic enhancer may vary within a 3-hour dosing interval.

In one embodiment, the unit dosage form of the invention is a single dosage form for oral administration containing all the at least one CCR5 antagonist, the at least one HIV-1 protease inhibitor and the at least one pharmacokinetic enhancer.

In one embodiment, the unit dosage form of the invention is a single or multiple dosage form for oral administration containing the at least one CCR5 antagonist, the at least one HIV-1 protease inhibitor and the at least one pharmacokinetic enhancer in a physically separated form. This may for example be realized by having three different dosage forms, the first containing the at least one CCR5 antagonist, the second containing either one of the at least one HIV-1 protease inhibitor and the at least one pharmacokinetic enhancer and the third one containing at least the other one of the at least one HIV-1 protease inhibitor and the at least one pharmacokinetic enhancer.

In one embodiment, the dosage form of the invention is a solid oral formulation such as a tablets, capsules containing particulates, liquids, or powders, lozenges (including liquid-filled), chews, multi- and nano-particulates, gels, solid solution, liposome, films (including muco-adhesive), ovules, sprays and liquid formulations. Preferably the unit dosage form of the invention is a tablet or capsule.

In one embodiment the dosage form of the invention is a liquid oral formulation such as suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

In another aspect, the present invention encompasses pharmaceutical compositions for oral administration to a treatment-naïve patient infected with CCR5 tropic HIV-1 virus, such composition comprising a therapeutically effective amount of at least one CCR5 antagonist, at least one HIV-1 protease inhibitor and at least one pharmacokinetic enhancer and one or more pharmaceutically acceptable excipients, carriers and/or diluents. Preferably, the term “excipient” is used herein to describe any ingredient other than the active compounds of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences', 19th Edition (Mack Publishing Company, 1995).

The compounds of the invention are generally administered orally. Oral administration may involve swallowing, so that the compounds enter the gastrointestinal tract, or buccal or sublingual administration may be employed by which the compounds enter the blood stream directly from the mouth.

The compounds of the invention may also be used in fast-dissolving, fast-disintegrating dosage forms such as those described in Expert Opinion in Therapeutic Patents, 11 (6), 981-986 by Liang and Chen (2001).

For tablet or capsule dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolat, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate. Tablets may also optionally include surface active agents, such as sodium lauryl sulfate and polysorbate 80, and glidants such as silicon dioxide and talc. When present, surface active agents may comprise from 0. 2 wt % to 5 wt % of the tablet, and glidants may comprise from 0.2 wt % to 1 wt % of the tablet. Tablets also generally contain lubricants such as magnesium stearate, calcium stearate, zinc stearate, sodium stearyl fumarate, and mixtures of magnesium stearate with sodium lauryl sulfate. Lubricants generally comprise from 0.25 wt % to 10 wt %, preferably from 0.5 wt % to 3 wt % of the tablet. Other possible ingredients include antioxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated. The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration are typically formulated for immediate release. Modified release forms are also possible. Other suitable release technologies such as high energy dispersions and osmotic and coated particles can be found in Verma et al, Pharmaceutical Technology On-line, 25 (2), 1-14 (2001). The use of chewing gum to achieve controlled release is described in WO 00/35298.

In another aspect, the present invention relates to a kit comprising a therapeutically effective amount of at least one CCR5 antagonist, at least one HIV-1 protease inhibitor and at least one pharmacokinetic enhancer as a combined preparation for simultaneous, separate or sequential oral administration to a treatment-naïve patient infected with CCR5 tropic HIV-1 virus. In the kit the composition(s) containing the various ingredients may conveniently be combined for co-administration.

In one embodiment, the kit of the invention comprises
one or more discrete pharmaceutical compositions,
   at least one of which contains the at least one CCR5 antagonist and
   at least another one which contains the at least one HIV-1 protease inhibitor and the at least one pharmacokinetic enhancer, preferably one discrete composition for each of the two,
      either in a combined or physically separated form, and
means for separately retaining said compositions, such as a container, divided bottle, or divided foil packet.

In one embodiment of the invention, the kit comprises at least three physically distinct compositions, one containing the at least one CCR5 antagonist, one containing the first of the at least one HIV-1 protease inhibitor and the at least one pharmacokinetic enhancer and one containing the second and more of the at least one HIV-1 protease inhibitor and the at least one pharmacokinetic enhancer. Preferably, the HIV-1 protease inhibitor and the pharmacokinetic enhancer are contained in physically distinct compositions.

An example of the kits discussed above is the familiar blister pack used for the packaging of tablets, capsules and the like.

The kit of the invention is particularly suitable for administering the separate compositions at different dosage intervals. To assist compliance, the kit typically comprises directions for administration and may be provided with a so-called memory aid.

In another aspect, the present invention relates to a method of treating a disorder selected from the group consisting of HIV-1 infection, retroviral infections genetically related to HIV, and AIDS, said method comprising simultaneous, separate or sequential oral administration of a therapeutically effective amount of at least one CCR5 antagonist, at least one HIV-1 protease inhibitor and at least one pharmacokinetic enhancer to a treatment-naïve patient infected with CCR5 tropic HIV-1 virus.

It is understood that all the embodiments and features disclosed above, notably regarding dose regimens, amounts of the ingredients which are administrable die as well as order and timing of administration of the single ingredients, apply to the method aspect.

The method of treatment of the present invention can be performed by relying on the kit and/or dosage unit forms disclosed above.

By means of the invention presently disclosed, a viral inhibition similar to conventional dual NRTIs HIV therapy is this now achievable but with lower side effects, lower potential for viral resistance development and potential for use in people with virus resistant to reverse transcriptase inhibitors (including non nucleoside inhibitors). Additionally, in certain embodiments of the invention a once per day (QD) HIV-1 effective regimen has been implemented leading to further advantages in terms of simplified dose regimen and an enhanced adherence.

Further embodiments and advantages of the present invention will become apparent to a skilled reader in light of the examples provided below.

### EXAMPLE 1

### Pharmacokinetics of Once Daily Maraviroc Co-Administered with Atazanavir/Ritonavir in Treatment-naïve HIV-infected Patients

Maraviroc (MVC) is primarily cleared by metabolism via CYP3A4. PK modelling studies (performed internally and not published) suggest that ATV/r, a potent CYP3A4 inhibitor, may make it possible to dose MVC once daily. In the pivotal Phase 3 MOTIVATE studies, where maraviroc was given once- or twice-daily with an optimized background regimen to treatment-experienced patients, the intersubject variability in the average concentrations (C_{avg}) of MVC was thought to be largely influenced by background therapy.

This PK substudy has been designed to examine the PK of MVC 150 mg once daily in combination with ATV/r, without confounding effects of other background therapy. Based on exposure-response analysis from the treatment-naive MERIT study, where maraviroc was dosed twice-daily with zidovudine/lamivudine, near maximal efficacy with MVC is achieved at a C_{avg} above approximately 75 ng/mL.

Treatment-naïve patients (N=121) were randomized 1:1 to either receive MVC 150 mg QD or tenofovir/emtricitabine 300/200 mg (Truvada®) QD both in combination with ATV/r 300/100 mg QD for 48 weeks. A subset of 15 patients in the MVC treatment arm at participating US sites was included in this PK substudy. Blood plasma samples were collected at predose and at 0.5, 1, 2, 3, 4, 6, 8, 10, and 24 hours postdose at Week 2. Based on the individual plasma concentration-time data, using actual sampling times, the AUC₂₄ₕ, C_{avg} (AUC/T), Cₘₐₓ, and Cₘᵢₙ, at Week 2 were determined by non-compartmental analyses and summary statistics were determined. 15 subjects, all men, were enrolled in the PK substudy; 11 were White, 3 were Black and 1 subject was of mixed race. The PK data [median (range)] for MVC when dosed once daily in combination with ATV/r at Week 2 were as follows: AUC₂₄=4330 ng·h/mL (1920-7310); C_{avg}=180 ng/mL (80-305); Cₘₐₓ=650 ng/mL (178-1490); Cₘᵢₙ=37.0 ng/mL (8.4-92.7). All 15 subjects achieved the targeted MVC C_{avg} (≧75 ng/mL) for near maximal virologic efficacy based on the exposure-response analysis from the MERIT study.

This data fully confirms that ATV/r makes it possible to dose MVC at 150 mg die once per day and thereby reaching targeted MVC C_{avg} (≧75 ng/mL) which provides for the near maximal efficacy of MVC as determined in the MERIT study.

### EXAMPLE 2

To ascertain whether a once daily, nucleoside-sparing regimen using a CCR5 antagonist could be safely and efficaciously administered to HIV-positive patients infected with CCR5-tropic HIV, a randomized, controlled study is being conducted. In this study, HIV-positive patients who had never been treated before, who had a virus that used the CCR5 co-receptor and that did not have any resistance mutations were randomized to receive atazanavir (300 mg QD) and ritonavir 100 mg QD with either maraviroc (150 mg QD) or Truvada.

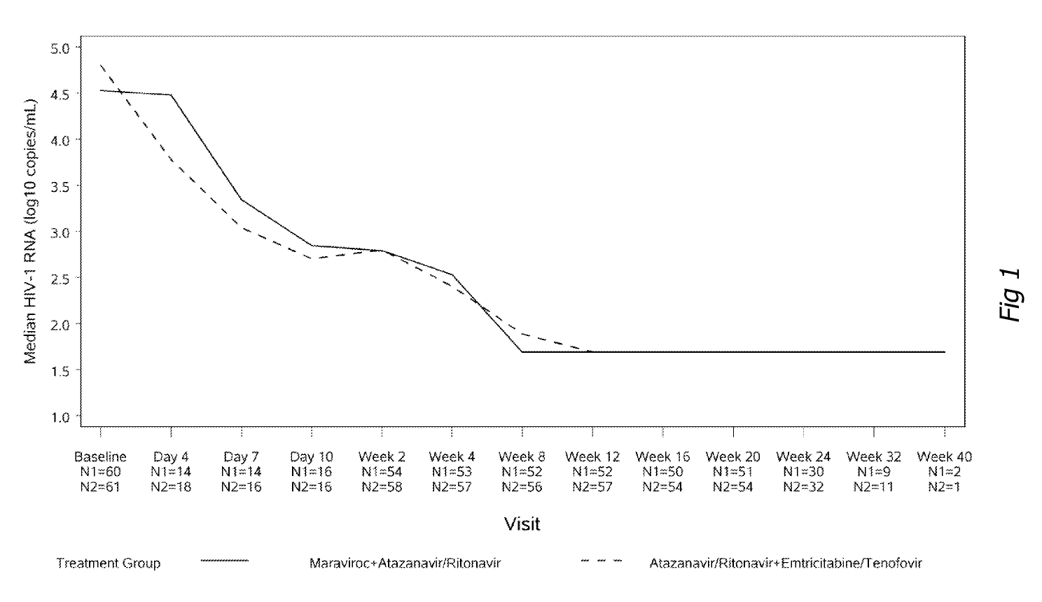

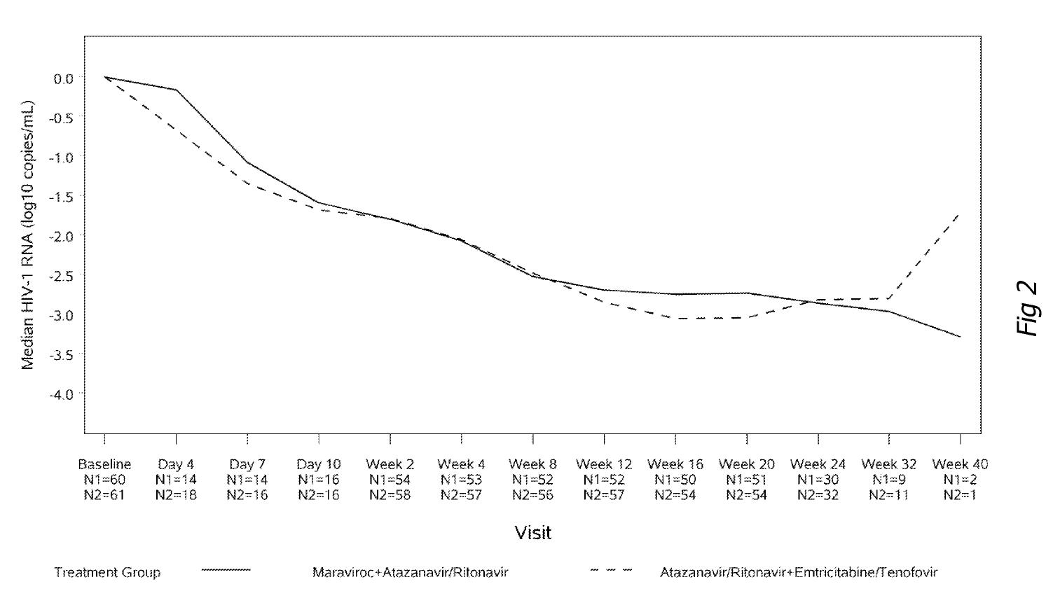

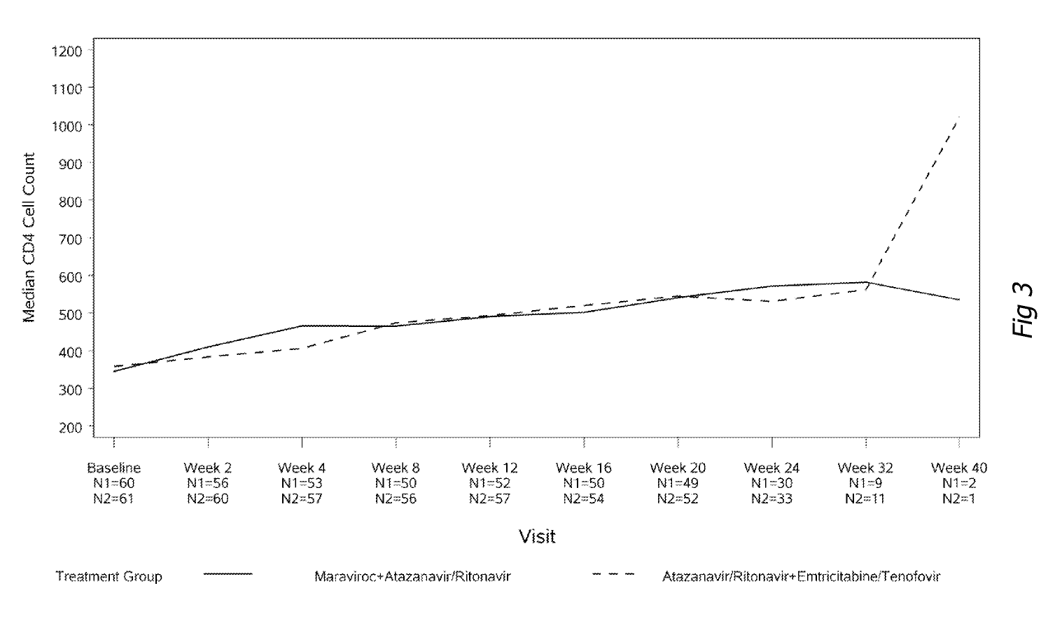

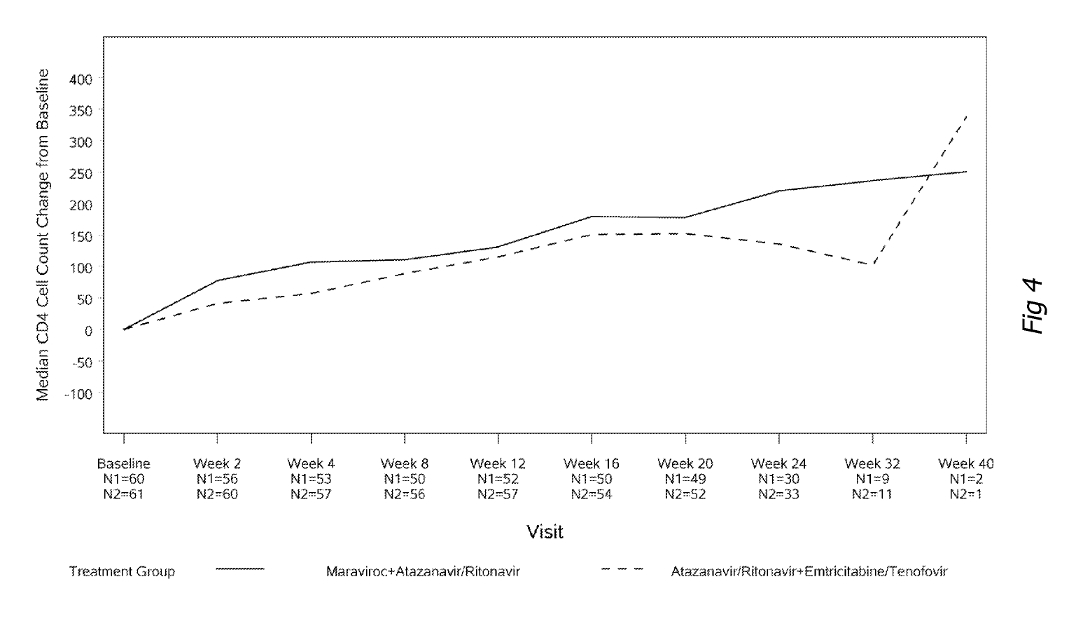

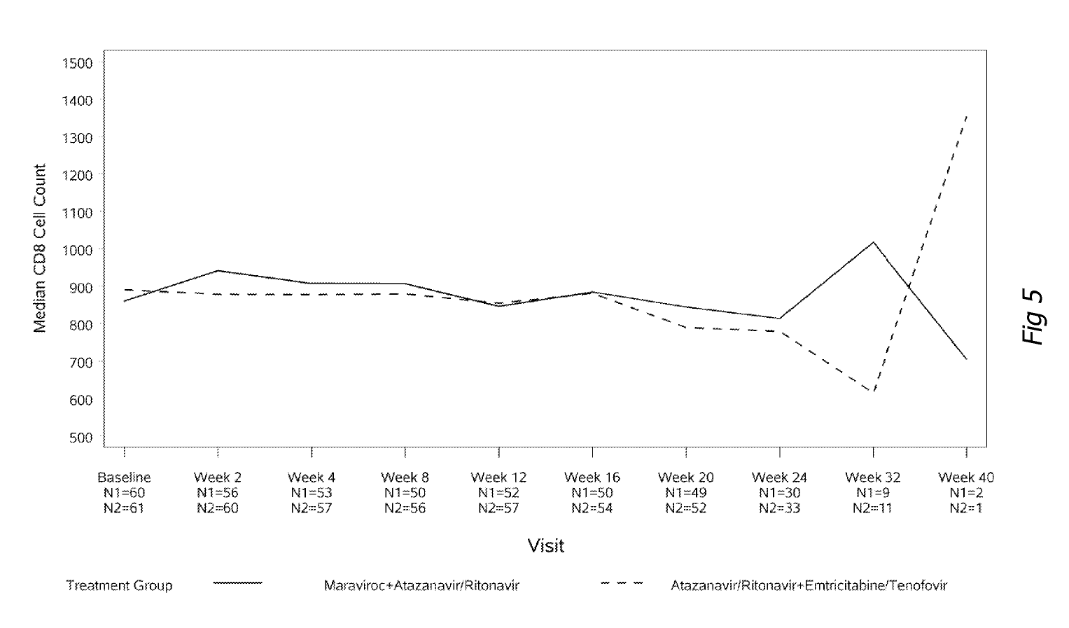

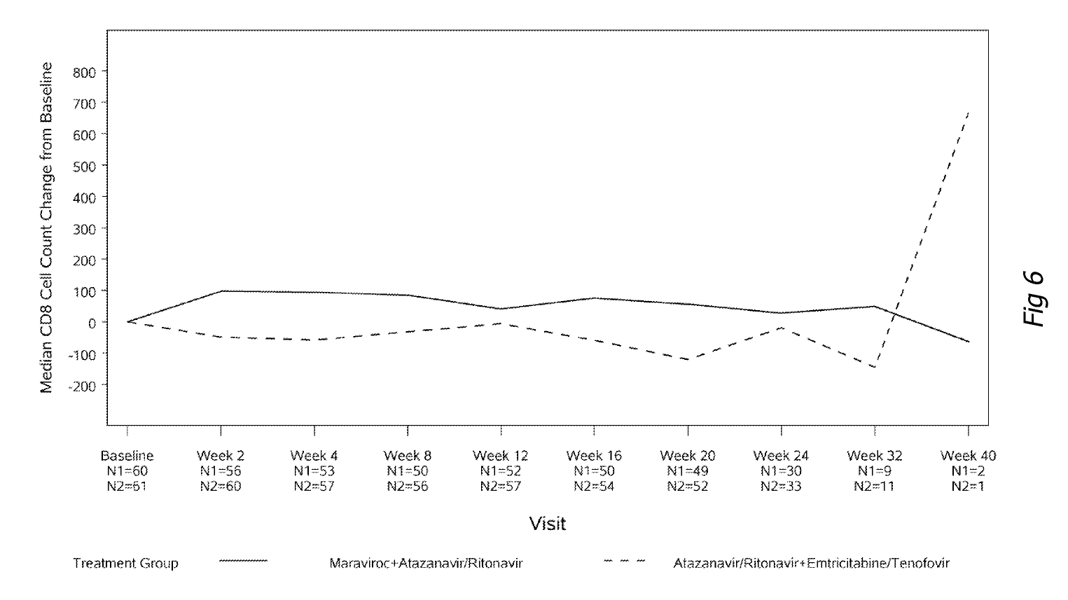

One hundred and twenty-one patients enrolled into the study; 60 in the maraviroc arm and 61 in the Truvada arm. Baseline characteristics were similar in patients in both treatment groups. Results after 24 weeks of observation are available and reported in FIGS. 1 to 6. The percentage of patients who achieved less than 400 and less than 50 copies/mL at week 24 (which are accepted treatment objectives of HIV therapy) were 93% and 90% (Truvada and maraviroc) and 89% and 80% (Truvada and maraviroc) respectively. Patients who received maraviroc experienced an increase in CD4+ of 195 cells, whereas patients who received Truvada experienced an increase of 173 cells. There were 4 discontinuations in the maraviroc arm and 3 in the Truvada arm. The safety profile of the regimens was comparable.

This pilot study demonstrates that the experimental once daily nucleoside sparing regimen is, over 24 weeks of observation, as safe and effective as the standard of care of atazanavir and ritonavir plus Truvada. The divergence on the right sides of the curves of FIG. 1-6 is due to still low number of subjects at late stages of the trial.

What is claimed is:

1. A combination comprising a therapeutically effective amount of at least one CCR5 antagonist, at least one HIV-1 protease inhibitor and at least one pharmacokinetic enhancer of said at least one CCR5 antagonist and/or at least one HIV-1 protease inhibitor for use in oral treatment of a disorder selected from the group consisting of HIV-1 infection, retroviral infections genetically related to HIV, and AIDS, in a treatment-naïve patient infected with CCR5 tropic HIV-1 virus.

2. The combination according to claim 1, comprising a therapeutically effective amount of at least one CCR5 antagonist and at least two HIV-1 protease inhibitors for use in oral treatment of a disorder selected from the group consisting of HIV-1 infection, retroviral infections genetically related to HIV, and AIDS, in a treatment-naïve patient infected with CCR5 tropic HIV-1 virus.

3. The combination according to claim 1 or claim 2, wherein no HIV-1 nucleotide reverse transcriptase inhibitors (NRTIs) are contained.

4. The combination according to claim 1 any one or more of the preceding claims, wherein the at least one CCR5 antagonist is selected from the group consisting of maraviroc, NCB-9471, PRO-140, CCR5mAb004, TAK-779, ZM-688523, 4-chloro-6-fluoro sulphonamide,TAK-220, TAK-652, SC-351125, ancriviroc, vicriviroc, PRO-140, aplaviroc, AMD-887, INC-B9471, CMPD-167, methyl1-endo-{8-[(3S)-3-(acetylamino)-3-(3-fluorophenyl) propyl]-8-azabicyclo[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo [3.2.1]oct-3-yl}-2-methyl-4,5,6,7-tetrahydro-3H-imidazo[4,5-c]pyridine-5-carboxylate, ethyl 1-endo-{8-[(3S)-3-(acetylamino)-3-(3-fluorophenyl)propyl]-8-azabicyclo[3.2.1]oct-3-yl}-2-methyl-4, 5,6,7-tetrahydro-1H-imidazo[4,5-c]pyridine-5-carboxylate and N-{(1S)-3-[3-endo-(5-Isobutyryl-2-methyl-4,5,6, 7-tetrahydro-1H-imidazo[4,5-c]pyridin-1-yl)-8-azabicyclo[3.2.1]oct-8-yl]-1-(3-fluorophenyl)propyl}acetamide) and pharmaceutically acceptable salts or solvates or solvates thereof.

5. The combination according to claim 1, wherein the at least one CCR5 antagonist comprises maraviroc.

6. The combination according to claim 1, wherein the at least one HIV-1 protease inhibitor is selected from the group consisting of saquinavir, indinavir, nelfinavir, amprenavir, lopinavir, atazanavir, fosamprenavir, tipranavir and darunavir and wherein the at least one pharmacokinetic enhancer is ritonavir.

7. The combination according to claim 1, comprising maraviroc at an amount between about 150 and about 300 mg, ritonavir at an amount between about 100 and about 200 mg and atazanavir at an amount of about 300 mg.

8. The combination according to claim 1, comprising maraviroc 150 mg, atazanavir 300 mg and ritonavir 100 mg for use in a once per day (QD) regimen oral treatment.

9. (canceled)

10. (canceled)

11. A pharmaceutical composition for oral administration to a treatment-naïve patient infected with CCR5 tropic HIV-1 virus, said composition comprising a combination as defined in claim 1 and one or more pharmaceutically acceptable excipients, carriers and/or diluents.

12. A unit dosage form comprising a combination as defined in claim 1 for use in oral treatment of a disorder selected from the group consisting of HIV-1 infection, retroviral infections genetically related to HIV-1, and AIDS, in a treatment-naïve patient infected with CCR5 tropic HIV-1 virus.

13. The unit dosage form according to claim 12 as a single or multiple dosage form for oral administration containing the at least one CCR5 antagonist and the at least two HIV-1 protease inhibitors in a physically separated form.

14. A kit comprising a combination as defined in claim 1 as a combined preparation for simultaneous, separate or sequential oral administration to a treatment-naïve patient infected with CCR5 tropic HIV-1 virus.

15. The kit according to claim 14 comprising at least three physically distinct compositions, one containing the at least one CCR5 antagonist, one containing the at least one HIV-1 protease inhibitor and one containing the at least one pharmacokinetic enhancer.

16. A method of treating a disorder selected from the group consisting of HIV-1 infection, retroviral infections genetically related to HIV, and AIDS, said method comprising simultaneous, separate or sequential oral administration of a combination as defined in claim 1 to a treatment-naïve patient infected with CCR5 tropic HIV-1 virus.
